(19) United States
(12) Patent Application Publication
(10) Pub. No.: US 2020/0093394 A1
(43) Pub. Date: Mar. 26, 2020

(54) SYSTEMS FOR AN MRI BREAST RF COIL ARRAY

(71) Applicant: General Electric Company, Schenectady, NY (US)

(72) Inventors: Jun LI, Beijing (CN); Jiabin YAO, Beijing (CN)

(21) Appl. No.: 16/468,870

(22) PCT Filed: May 2, 2017
(86) PCT No.: PCT/US2017/030654

§ 371 Date: Jun. 12, 2019

Related U.S. Application Data
(60) Provisional application No. 62/433,718, filed on Dec. 13, 2016.

(51) Int. Cl. A61B 5/055; G01R 33/341; G01R 33/36; G01R 33/56; A61B 5/00

(52) U.S. Cl. CPC A61B 5/055; G01R 33/341; G01R 33/3614; A61B 5/004; A61B 5/6831; A61B 5/6804; G01R 33/56

(57) ABSTRACT

Various methods and systems are provided for a flexible, comfortable breast radio frequency (RF) coil assembly for a magnetic resonance imaging system. The breast RF coil assembly may include two cups each housing eight RF coils arranged in an overlapping fashion. The breast RF coil assembly may further include respective flanking coil arrays on a side of each cup, with each flanking coil array housing four RF coils.

### CROSS REFERENCE TO RELATED APPLICATIONS

The present application claims priority to U.S. Provisional Application No. 62/433,718, entitled “SYSTEMS FOR AN MRI BREAST RF COIL ARRAY,” and filed on Dec. 13, 2016, the entire contents of which are hereby incorporated by reference for all purposes.

### FIELD

Embodiments of the subject matter disclosed herein relate to magnetic resonance imaging (MRI), and more particularly, to MRI radio frequency (RF) coils.

### BACKGROUND

Magnetic resonance imaging (MRI) is a medical imaging modality that can produce images of an interior of a patient without x-ray radiation or other types of ionizing radiation. MRI systems utilize a superconducting magnet to create a strong, uniform, static magnetic field within a designated region (e.g., within a passage shaped to receive a patient). When a body of a patient (or portion of the body of the patient) is positioned within the magnetic field, nuclear spins associated with the hydrogen nuclei that form water within tissues of the patient become polarized. The magnetic moments associated with these spins become aligned along the direction of the magnetic field and result in a small net tissue magnetization in the direction of the magnetic field. MRI systems additionally include magnetic gradient coils that produce spatially-varying magnetic fields of smaller magnitudes relative to a magnitude of the uniform magnetic field resulting from the superconducting magnet. The spatially-varying magnetic fields are configured to be orthogonal to each other in order to spatially encode the region by creating a signature resonance frequency of the hydrogen nuclei at different locations in the body of the patient. Radio frequency (RF) coil assemblies are then used to create pulses of RF energy at or near the resonance frequency of the hydrogen nuclei. The pulses of RF energy are absorbed by the hydrogen nuclei, thereby adding energy to the nuclear spin system and adjusting the hydrogen nuclei from a rest state to an excited state. As the hydrogen nuclei relax back to the rest state from the excited state, they release the absorbed energy in the form of an RF signal. This signal is detected by the MRI system and is transformed into an image by a computer using known reconstruction algorithms.

MRI may provide higher sensitivity relative to other imaging modalities (e.g., radiography) when utilized to scan patient breast tissues. However, current breast tissue MRI scanning configurations may not have widespread usage. For example, a scan time to image breast tissue via MRI may be longer than a scan time to image other parts of a patient's body via MRI due to the need to perform separate scans for multi-plan, bilateral, and single breast images in a breast tissue MRI examination. The increased scan times may not be suitable for certain clinical applications. Additionally, current RF coil assemblies may not be shaped to comfortably conform to the body of a patient, thereby lowering patient satisfaction and increasing a distance between breasts of the patient and RF coils included within the assemblies. The increased distance may result in degradation of images produced by the MRI system, such as a reduced signal-to-noise ratio (SNR) of images produced by the MRI system. Further still, some RF coil assemblies may include eight or less RF coils, thereby further reducing the SNR compared to assemblies which include a larger number of RF coils. It is therefore desirable to provide an RF coil assembly shaped to comfortably support breasts of a patient and configured to enable bilateral scanning of the patient with an increased number of coils.

### BRIEF DESCRIPTION

In one embodiment, an RF coil for a medical imaging device includes a first coil array housed in a first cup-shaped support structure; a second coil array housed in a second cup-shaped support structure; a third coil array flanking the first coil array; and a fourth coil array flanking the second coil array.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 shows a schematic diagram of an example magnetic resonance imaging (MRI) system including a radio frequency (RF) coil assembly.

FIGS. 2A-2B show different views of a first embodiment of an RF coil assembly including a plurality of coil arrays.

FIGS. 3A-3B show different views of a second embodiment of an RF coil assembly including a plurality of coil arrays.

FIG. 4A shows the coil arrays of FIGS. 2A-2B removed from their corresponding RF coil assembly.

FIG. 4B shows a coil array of FIGS. 3A-3B removed from its corresponding RF coil assembly.

FIGS. 5A-5B show cross-sectional views of an RF coil assembly in a contracted state and in an expanded state.

FIG. 6 shows a side view of a patient wearing the RF coil assembly of FIGS. 2A-2B.

FIG. 7 schematically shows an embodiment of a signal-receiver section of a magnetic resonance imaging (MRI) system including an RF coil assembly.

FIG. 8 schematically shows an RF coil of the RF coil assembly shown by FIG. 7, with the RF coil coupled to a pre-amplifier.

FIG. 9 schematically shows components of the pre-amplifier shown by FIG. 8.

### DETAILED DESCRIPTION

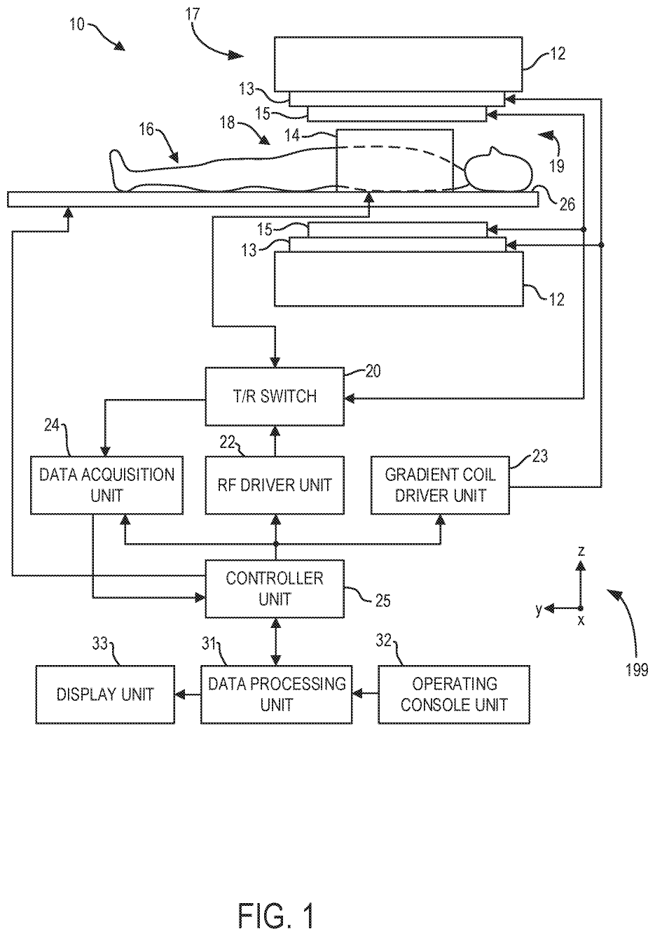

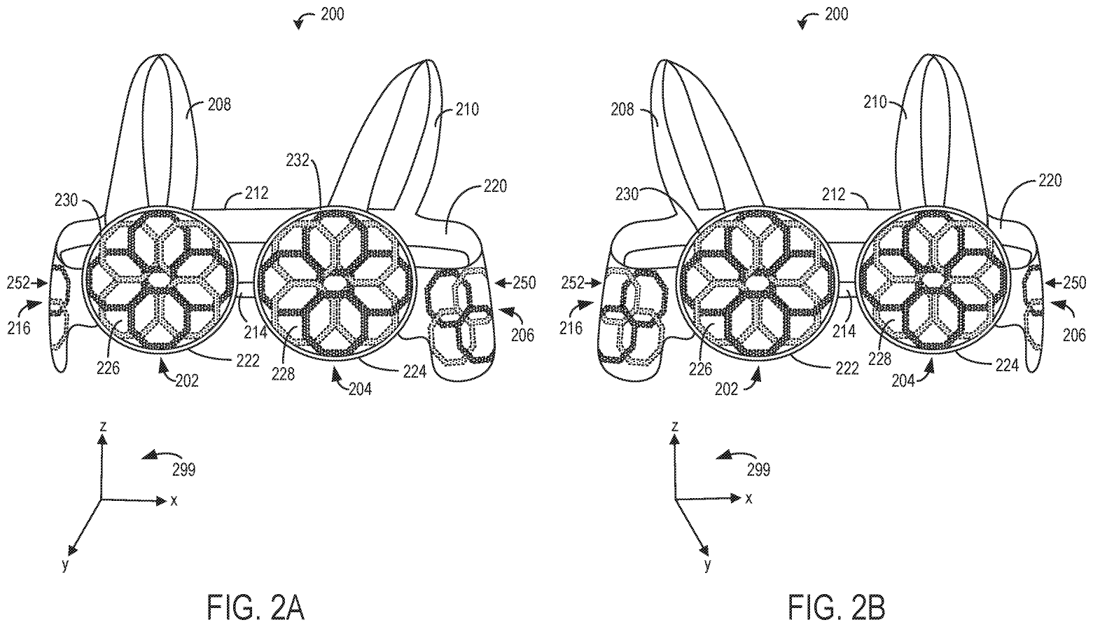

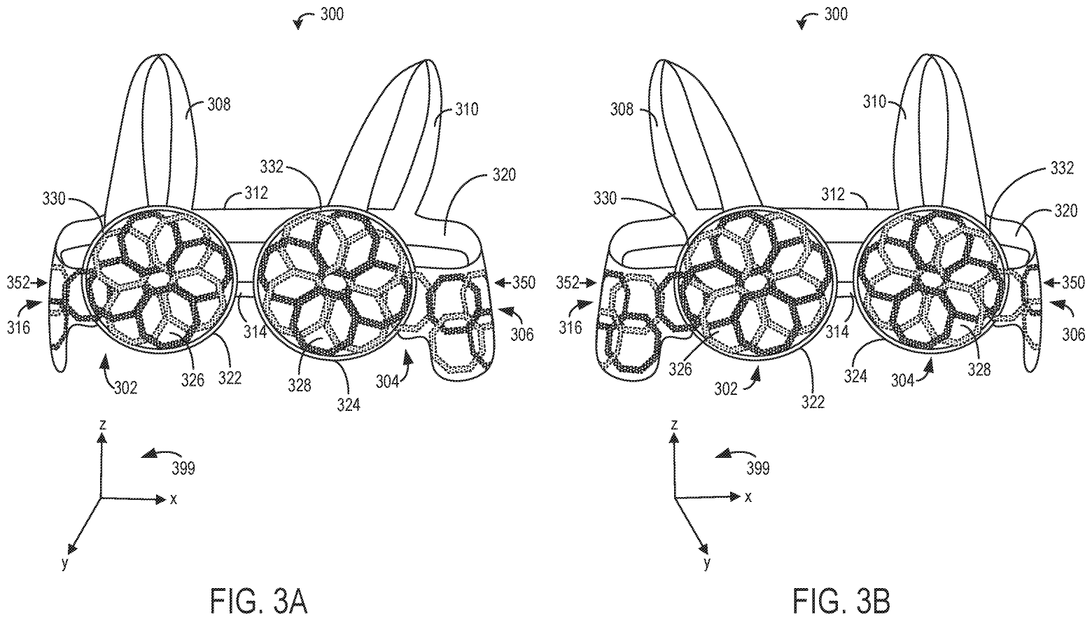

The following description relates to various embodiments of a radio frequency (RF) coil assembly. In particular, an RF coil assembly is provided for a magnetic resonance imaging (MRI) system (such as the MRI system shown by FIG. 1). The RF coil assembly (such as the RF coil assemblies shown by FIGS. 2A-2B and FIGS. 3A-3B) is shaped to conform to a body of a patient and includes a plurality of RF coil arrays positioned in an adjustable, bra-like support structure. A first RF coil array and a second RF coil array may each include eight RF coils coupled to respective cups of the bra-like support structure. By including eight RF coils in each of the first RF coil array and the second RF coil array, a distance between adjacent RF coils within the RF coil arrays may be reduced and the RF coils may be positioned closer to the anatomy being imaged. A signal-to-noise (SNR) ratio of images produced by the MRI system may thereby be increased and a larger amount of the patient's body may be scanned without repositioning the patient and/or the RF coil assembly.

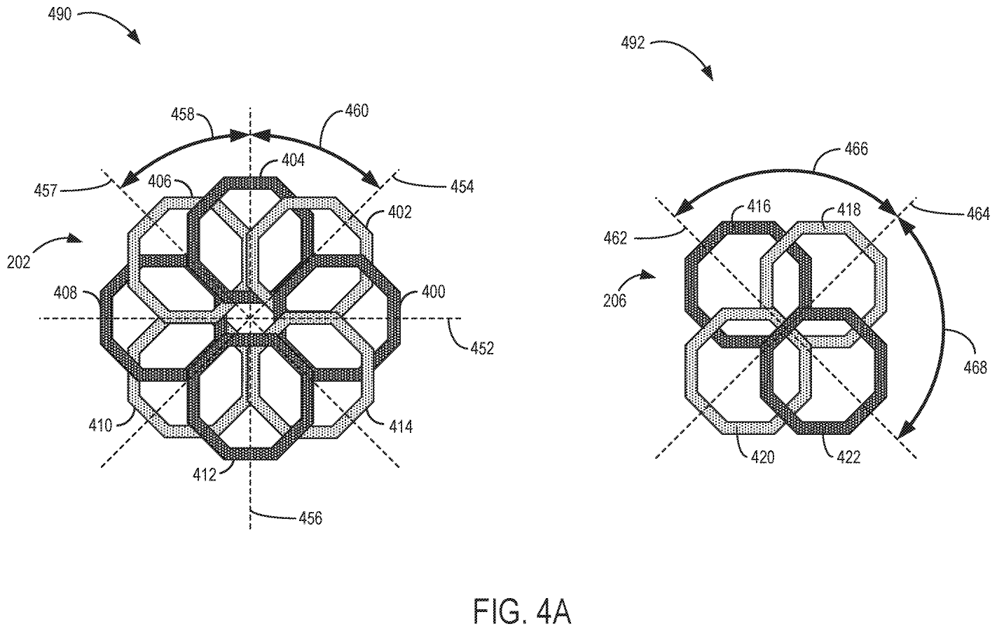

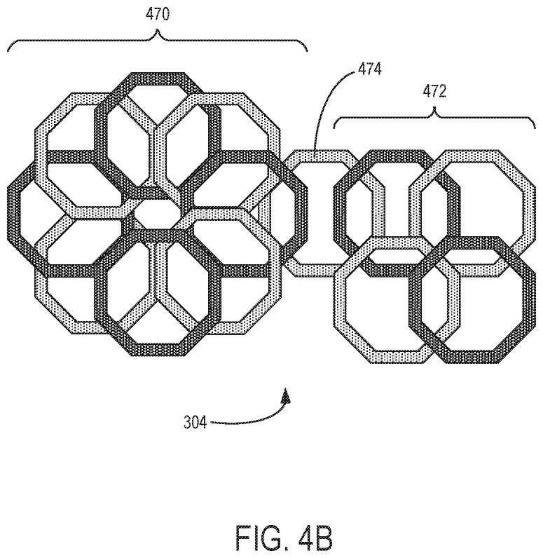

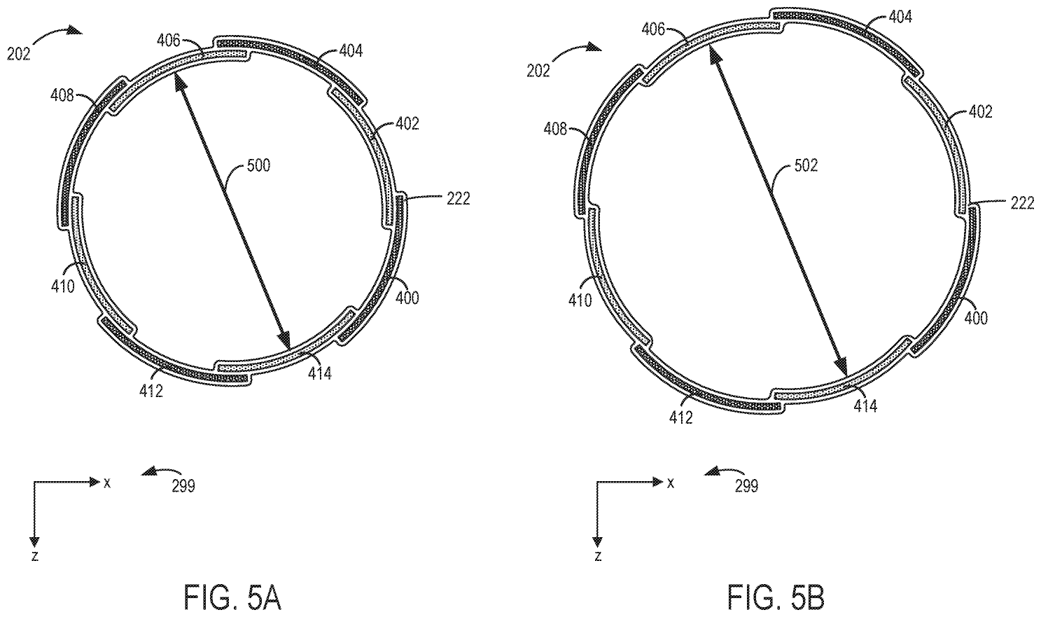

The RF coils within each of the first RF coil array and second RF coil array may be arranged in a semi-overlapping structure as shown by FIGS. 4A-4B and FIGS. 5A-5B. In one example, the RF coils within the first RF coil array and second RF coil array may be positioned in an arrangement similar to petals of a lotus flower. By coupling the RF coil arrays to the cups of the bra-like support structure (e.g., one RF coil array for each cup), the RF coils of the RF coil arrays and the associated support structure may be flexible and compatible for imaging patients in a supine position or a prone position, thus increasing patient comfort. The cups may be made of stretchable, flexible, and/or soft material, and the RF coils within the RF coil arrays may be moveable relative to each other (as shown by FIGS. 5A-5B) during expansion and/or contraction of the cups, thereby enabling a diameter of the cups to expand or contract to conform to the body of the patient.

In a first embodiment, the RF coil assembly includes additional coil arrays positioned at side regions of a patient's chest and adjacent to respective cups of the bra-like support structure. Specifically, the RF coil assembly includes a third RF coil array and a fourth RF coil array (as shown by FIGS. 2A-2B and FIG. 4A). The third RF coil array and fourth RF coil array may each include four RF coils. In a second embodiment (as shown by FIGS. 3A-3B and FIG. 4B), the first RF coil array and second RF coil array are instead shaped to include extensions that additionally enclose the side regions of the patient's chest and include additional RF coils positioned at the side regions. In this way, the RF coils positioned at the side regions (e.g., via the third RF coil array and fourth RF coil array of the first embodiment, or the extensions of the first RF coil array and second RF coil array of the second embodiment) further increase an amount of the patient's body that may be scanned without repositioning the patient and/or the RF coil assembly, thereby decreasing an amount of time to perform the scan and increasing an amount of tissue that may be scanned (such as lymph nodes positioned at the sides of the patient's body).

FIG. 1 illustrates an MRI system 10 that includes a magnetostatic field magnet unit 12 (e.g., a superconducting magnet), a magnetic gradient generator 13, local RF coils 14 (which may be referred to collectively herein as an RF coil assembly), volumetric RF coils 15, a transmit/receive (T/R) switch 20, an RF signal driver 22, a gradient driver 23, a data acquisition unit 24, a controller unit 25, a patient table 26 (which may be referred to herein as a bed), a data processing unit 31, an operating console unit 32, and a display unit 33. The local RF coils 14 are surface coils configured to be placed proximate to surfaces of an anatomical structure (e.g., a breast) of a subject 16 (e.g., a patient) to be scanned by the MRI system 10. The volumetric RF coils 15 are coils configured to transmit RF signals (e.g., electromagnetic waves at radio frequencies), and the local RF coils 14 are configured to receive RF signals. As such, the volumetric RF coils 15 and the local RF coils 14 are spatially separated from each other but may be electromagnetically coupled to each other. In some examples, the local RF coils 14 may both transmit and receive RF signals. Example operational modes of the coils (e.g., local RF coils 14 and volumetric RF coils 15) are described further below.

The MRI system 10 includes the patient table 26 for placing a subject 16 (e.g., a patient) thereon. The subject 16 may be moved inside and outside of an imaging space 18 by moving the patient table 26. The imaging space 18 may be positioned within a bore 19 of a gantry 17 formed by the MRI system 10. In some examples, controller unit 25 may transmit control signals (e.g., electrical signals) to operating console unit 32 and/or display unit 33 in order to indicate a position of the patient table 26 within the imaging space 18 to an operator (e.g., user, technician, etc.) of the MRI system 10.

The operating console unit 32 includes user input devices such as a keyboard and a mouse. The operating console unit 32 is utilized by an operator, for example, to input an imaging protocol (e.g., a parallel imaging protocol) and to set a region where an imaging sequence is to be executed. Data input by the operator into the operating console unit 32 about the imaging protocol and the imaging sequence execution region is output to the controller unit 25.

The display unit 33 includes a graphical display device (e.g., computer screen) and displays an image on the graphical display device based on control signals received from the controller unit 25. The display unit 33 displays, for example, an image regarding an input item about which the operator inputs operation data from the operating console unit 32. The display unit 33 also displays a slice image of the subject 16 generated by the data processing unit 31.

The data processing unit 31 includes a computer and a recording medium (e.g., hard drive) on which a program to be executed by the computer to perform predetermined data processing is recorded. The data processing unit 31 is electrically coupled with the controller unit 25 and performs data processing based on control signals received from the controller unit 25. The data processing unit 31 is also connected to the data acquisition unit 24 and generates spectrum data by applying various image processing operations to magnetic resonance (MR) signals output from the data acquisition unit 24 (described in further detail below).

The magnetostatic field magnet unit 12 includes an annular superconducting electromagnet coupled to a toroidal vacuum vessel (e.g., gantry 17) and positioned within an interior of the toroidal vacuum vessel. The electromagnet defines a cylindrical space (e.g., bore 19) surrounding the subject 16, and generates a magnetostatic field of approximately constant magnitude and direction within the cylinder space (e.g., in a direction of the y-axis within the cylinder space as shown by reference axes 199). The magnetostatic field generated by the electromagnet may be referred to herein as a uniform magnetic field.

The MRI system 10 also includes the magnetic gradient generator 13 that generates an additional magnetic field (which may be referred to herein as a gradient magnetic field) in the imaging space 18 so as to associate the MR signals received by the local RF coils 14 with three-dimensional positional information. For example, the gradient magnetic field produced by the magnetic gradient generator 13 may have a different magnitude (e.g., a different field strength) at different locations within the imaging space 18. The magnetic gradient generator 13 includes three gradient coil systems. Each gradient coil system adjusts the magnitude of the gradient magnetic field along one of three perpendicular directions. For example, a first gradient coil system adjusts the magnitude of the gradient magnetic field in a frequency encoding direction, a second gradient coil system adjusts the magnitude of the gradient magnetic field in a phase encoding direction, and a third gradient coil system adjusts the magnitude of the gradient magnetic field in a slice selection direction. The frequency encoding direction, phase encoding direction, and slice selection direction may be defined based on input from a user (e.g., operator) of the MRI system 10 (e.g., via the operating console unit 32). More specifically, the magnetic gradient generator 13 adjusts the magnitude of the gradient magnetic field in the slice selection direction of the subject 16 in response to input from the operator. The local RF coils 14 then transmits an RF pulse to a selected slice of the subject 16 and energizes the slice (e.g., excites a spin of hydrogen nuclei within the selected slice of the subject 16). The magnetic gradient generator 13 adjusts the magnitude of the gradient magnetic field in the phase encoding direction of the subject 16 to phase encode MR signals emitted by the slice energized by the RF pulse. The magnetic gradient generator 13 then adjusts the magnitude of the gradient magnetic field in the frequency encoding direction of the subject 16 to frequency encode MR signals emitted by the slice excited by the RF pulse.

The gradient driver 23 drives the magnetic gradient generator 13 based on a control signal received from the controller unit 25 and thereby generates the gradient magnetic field in the imaging space 18. The gradient driver 23 includes three systems of driver circuits (not shown) corresponding to the three gradient coil systems included in the magnetic gradient generator 13 (as described above).

The RF coils of the MRI system 10 (e.g., local RF coils 14 and/or volumetric RF coils 15) may transmit electromagnetic pulse signals to the subject 16 positioned within the imaging space 18, with the uniform magnetic field and the gradient magnetic field extending through the imaging space 18. The local RF coils 14 are shaped, for example, to enclose the region to be imaged of the subject 16. In some examples, the local RF coils 14 may be referred to as surface coils or receiver coils. The MRI system 10 receives MR signals from the subject 16 (e.g., via the data acquisition unit 24 coupled to the RF coils) and processes the MR signals (e.g., via the data processing unit 31) in order to construct an image of a slice of the subject 16 based on the received MR signals.

For example, during conditions in which the subject 16 is positioned to be scanned by the MRI system 10 (e.g., during conditions in which the subject 16 is within the imaging space 18), spins of hydrogen nuclei within the tissues of the subject 16 may be aligned with initial magnetization vectors resulting from a combination of the uniform magnetic field and the gradient magnetic field. The local RF coils 14 transmit, based on a control signal from the controller unit 25, an RF pulse that is an electromagnetic wave to the subject 16. The RF pulses transmitted to the subject 16 generate a high-frequency magnetic field within the slice of the subject 16 to be imaged (e.g., as selected by the operator of the MRI system 10). The high-frequency magnetic field excites a spin of hydrogen nuclei in the slice of the subject 16 and aligns the spins with different magnetization vectors relative to the initial magnetization vectors. As the spins of the excited hydrogen nuclei in the slice of the subject 16 relax and return into alignment with the initial magnetization vectors, the local RF coils 14 receive electromagnetic waves generated from the tissues of the subject 16 as a MR signals.

In some examples, the volumetric RF coils 15 may alternately (or additionally) be utilized to generate a high-frequency magnetic field similar to that described above with reference to the local RF coils 14. For example, the volumetric RF coils 15 are positioned to enclose the imaging space 18 and may produce RF pulses in a direction orthogonal to the direction of the uniform magnetic field generated by the magnetostatic field magnet unit 12 within the imaging space 18 in order to excite the hydrogen nuclei of the subject 16. The volumetric RF coils 15 are fixedly attached and coupled to the MRI system 10, unlike the local RF coils 14 which may be disconnected from the MRI system 10 and replaced with different local RF coils. Furthermore, whereas local coils such as those comprising the local RF coils 14 may transmit to and/or receive signals (e.g., transmit RF signals and/or receive MR signals) from a localized region of the subject 16 (e.g., a particular anatomical structure or slice of the subject 16), the volumetric RF coils 15 may transmit to and/or receive signals from a larger portion of the subject 16 (e.g., an entire body of the subject 16).

In one example, utilizing local RF coils 14 to receive MR signals and volumetric RF coils 15 to transmit RF signals may increase a ratio of excited hydrogen nuclei relative to non-excited hydrogen nuclei and may result in increased image clarity. However, exciting the hydrogen nuclei via the volumetric RF coils 15 may result in a larger amount of power deposited into tissues of the subject 16 via the RF signals relative to a configuration in which RF signals are instead transmitted into the subject 16 via the local RF coils 14. During conditions in which the local RF coils 14 are instead utilized to both transmit RF signals into the tissues of the subject 16 and receive MR signals from the tissues, the local RF coils may reduce an amount of power deposited into the tissues of the subject 16 by directing the RF signals towards a particular anatomical structure or slice rather than the entire body of the patient. However, it should be appreciated that the particular configuration of the local RF coils 14 and/or the volumetric RF coils 15 (e.g., imaging the entire subject 16 via the volumetric RF coils 15, or imaging a smaller portion of the subject 16 via the local RF coils 14 as described above) depends on the clinical application for which the MRI system 10 is utilized.

The RF signal driver 22 electrically coupled to the coils (e.g., volumetric RF coils 15 and/or local RF coils 14) via the T/R switch 20 includes a gate modulator (not shown), an RF power amplifier (not shown), and an RF oscillator (not shown) that are used to drive the local RF coils 14 and/or volumetric RF coils 15 to form a high-frequency magnetic field in the imaging space 18 (as described above). The RF signal driver 22 modulates the RF signal received from the RF oscillator into a signal of predetermined timing and having a predetermined envelope via the gate modulator, with the RF signal based on a control signal from the controller unit 25. The RF signal modulated by the gate modulator is amplified by the RF power amplifier and then output to the local RF coils 14 and/or volumetric RF coils 15.

The T/R switch 20 can selectively electrically couple the local RF coils 14 and/or the volumetric RF coils 15 to the data acquisition unit 24 when operating in a receive mode, and to the RF signal driver 22 when operating in a transmit mode. During conditions in which the local RF coils 14 and the volumetric RF coils 15 are both used in a single scan (e.g., during conditions in which the local RF coils 14 are configured to receive MR signals and the volumetric RF coils 15 are configured to transmit RF signals), the T/R switch 20 may direct control signals from the RF signal driver 22 to the volumetric RF coils 15 while directing received MR signals from the local RF coils 14 to the data acquisition unit 24. As described above, the volumetric RF coils 15 may be configured to operate in a transmit-only mode, a receive-only mode, or a transmit-and-receive mode. The local RF coils 14 may be configured to operate in a transmit-and-receive mode or a receive-only mode.

The data acquisition unit 24 includes a preamplifier (not shown), a phase detector (not shown), and an analog/digital converter (not shown) used to acquire the magnetic resonance signals received by the local RF coils 14 and/or volumetric RF coils 15. In the data acquisition unit 24, the phase detector phase detects, using the output from the RF oscillator of the RF signal driver 22 as a reference signal, the MR signals received by the local RF coils 14 and/or volumetric RF coils 15 (with the MR signals being amplified by the preamplifier), and outputs the phase-detected analog MR signals to the analog/digital converter for conversion into digital signals. The digital signals thus obtained are output to the data processing unit 31 electrically coupled with the controller unit 25.

The controller unit 25 includes a computer and a recording medium on which a program to be executed by the computer is recorded. The program when executed by the computer causes various parts of the system to carry out operations corresponding to pre-determined scanning. The recording medium may comprise, for example, a read-only memory (ROM), flexible disk, hard disk, optical disk, magneto-optical disk, CD-ROM, or non-volatile memory card. The controller unit 25 is connected to the operating console unit 32 and processes the operation signals input to the operating console unit 32 (e.g., input by the operator of the MRI system 10) and furthermore controls the patient table 26, RF signal driver 22, gradient driver 23, and data acquisition unit 24 by outputting control signals to them. The controller unit 25 also controls, to obtain a desired image, the data processing unit 31 and the display unit 33 based on operation signals received from the operating console unit 32.

During a scan (e.g., imaging of the subject 16 according to the examples described above), coil-interfacing cables (not shown) may be used to transmit signals between the RF coils (e.g., local RF coils 14 and volumetric RF coils 15) and other aspects of the processing system (e.g., data acquisition unit 24, controller unit 25, and so on), for example to control the RF coils and/or to receive information from the RF coils. As explained previously, in one example the volumetric RF coils 15 may transmit RF signals and the local RF coils 14 may receive MR signals. The local RF coils 14 and/or volumetric RF coils 15 may include coils that are used to transmit RF excitation signals (“transmitter coil”) and coils that receive the MR signals emitted by an imaging subject (“receive coil”). In some examples, the transmitter coils and receive coils may be the same coils (e.g., configured to both transmit RF excitation signals and receive MR signals) such that the coils are a single mechanical structure or array of structures, with the transmit/receive modes of the coils switchable by auxiliary circuitry (e.g., T/R switch 20). In other examples, the volumetric RF coils 15 and local RF coils 14 may be independent structures that are physically coupled to each other via a data acquisition unit or other processing unit.

In some examples (e.g., examples in which the transmitter coils and receive coils are not the same coils), it may be desirable to configure the receive coils to be mechanically and electrically isolated from the transmitter coils in order to achieve an increased image quality. In one example, the receive coils (e.g., local RF coils 14) may be configured to receive MR signals for a duration following transmission of RF signals from the transmitter coils (e.g., volumetric RF coils 15). However, for the duration in which the transmitter coils are transmitting RF signals, it may be desirable to electromagnetically decouple the receive coils from the transmitter coils such that the receive coils are not resonant with the transmitter coils (e.g., such that the receive coils do not receive the RF signals from the transmitter coils). Electromechanically decoupling (e.g., deactivating) the receive coils during transmission of RF signals by the transmitter coils may reduce an amount of noise produced within auxiliary circuitry coupled to the receive coils and may result in increased image quality.

FIGS. 2A-2B each illustrate different perspective views of a first embodiment of an RF coil assembly 200 that is configured to be positioned over a patient's breasts during a breast MRI scan. In one example, the RF coil assembly 200 may be similar to the local RF coils 14 and may be utilized with an MRI system such as the MRI system 10 shown by FIG. 1 and described above. The coils of the RF coil assembly 200 may operate in various modes as described above with reference to the local RF coils 14 of FIG. 1 (e.g., receive-only or receive-and-transmit modes). Reference axes 299 are included by FIGS. 2A-2B for comparison of the views shown.

RF coil assembly 200 includes a first coil array 202, second coil array 204, third coil array 206, and a fourth coil array 216 coupled to a support structure 220. The coil arrays described herein may also be referred to as coil sets. In the example shown by FIGS. 2A-2B and described herein, the support structure 220 is a bra-like support structure including a first cup 222 and a second cup 224. The first cup 222 and second cup 224 may be referred to herein as cup-shaped support structures. The first cup 222 and second cup 224 are positioned at a front side of the support structure 220 and are each shaped to support breasts of a patient. The third coil array 206 is positioned at a first side 250 of the support structure 220 and the fourth coil array 216 is positioned at a second side 252 of the support structure 220. During conditions in which the RF coil assembly 200 is worn by a patient, the first side 250 of the support structure 220 and the second side 252 of the support structure 220 each surround the upper sides of the torso of the patient such that the third coil array 206 and fourth coil array 216 are positioned vertically beneath the axillae of the patient and proximate to the serratus anterior muscles (e.g., at the sides of the breasts of the patient). Additionally, the support structure 220 may include one or more straps (which may be referred to herein as support straps), such as first shoulder strap 208, second shoulder strap 210, back strap 212, and front strap 214. A length of one or more or each of the straps may be adjustable via hook and loop fasteners, buttons, or other fasteners in order to couple the support structure 220 to the patient. In one example, front strap 214 may couple first cup 222 to second cup 224, first shoulder strap 208 may couple first cup 222 to back strap 212, and second shoulder strap 210 may couple second cup 224 to back strap 212.

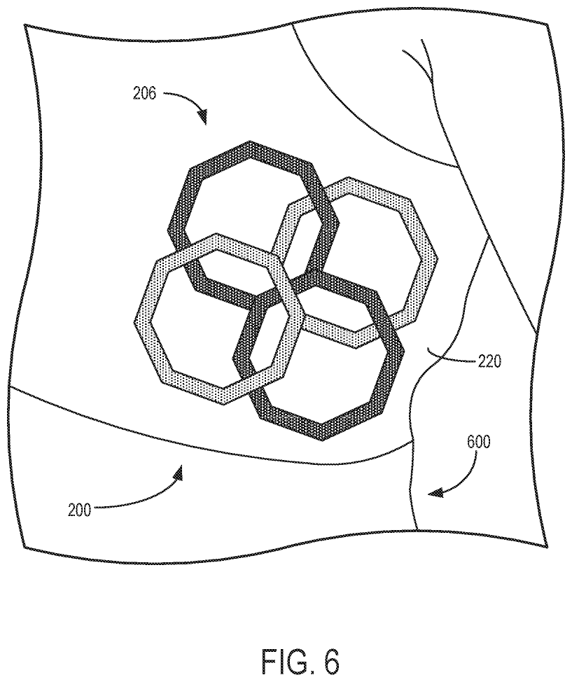

The first coil array 202 is coupled to the first cup 222 and the second coil array 204 is coupled to the second cup 224 such that the first coil array 202 and second coil array 204 are each positioned at a front end of the patient during conditions in which the support structure 220 is coupled to the patient (e.g., as shown by FIG. 6, with the support structure 220 coupled to an example patient 600). The third coil array 206 and fourth coil array 216 are each coupled with the support structure 220 such that the third coil array 206 and fourth coil array 216 are positioned at opposite sides of the patient during conditions in which the support structure 220 is coupled to the patient. In some examples, one or both of the third coil array 206 and fourth coil array 216 may be integrated as part of back strap 212. In other examples, one or both of the third coil array 206 and fourth coil array 216 may be coupled to back strap 212 via one or more additional straps. In yet further examples, the third coil array 206 and/or fourth coil array 216 may be coupled to the first cup 222 and/or second cup 224 (respectively) via one or more additional straps. The third coil array 206 and fourth coil array 216 may each enable MR signals to be received from multiple sides of the patient as described further below.

First coil array 202 and second coil array 204 each include a plurality of RF coils (described in further detail below with reference to FIG. 4A) coupled to their respective cup (e.g., first cup 222 and second cup 224) of the support structure 220. Second coil array 204 may be configured similarly to first coil array 202 (e.g., may include a similar number and/or relative arrangement of RF coils relative to the first coil array 202). Each cup is shaped to receive a breast of a patient such that MR signals emitted by a first breast of the patient are received substantially by the first coil array 202 and MR signals emitted by a second breast of the patient are received substantially by the second coil array 204. The first cup 222 and second cup 224 may each be formed of a stretchable or non-stretchable fabric (polyester, cotton, rayon, etc.), polymer (e.g., polyurethane), elastomer, foam, etc., or a combination thereof. The first cup 222 and second cup 224 may each include plastic or other polymer (non-metal) supports such as ribs, frames, etc.

As shown, first cup 222 coupled to first coil array 202 includes a substantially circular frame 230 supporting the cup-shaped exterior surface 226. Second cup 224 coupled to second coil array 204 includes a similar circular frame 232 supporting the cup-shaped exterior surface 228. The coils of the first coil array 202 and second coil array 204 may be sewn or otherwise secured to the respective exterior surface of the support structure 220 (e.g., exterior surface 226 of first cup 222 and exterior surface 228 of second cup 224, respectively). In some examples, the coils of one or more of the coil arrays may be exposed along an interior surface of the first cup 222 and/or second cup 224. In other examples, one or more layers of fabric or other material may be fastened to the interior surface of the first cup 222 and/or second cup 224 in order to contain the coils within their respective cups.

The third coil array 206 and fourth coil array 216 each include pluralities of RF coils similar to those included by the first coil array 202 and second coil array 204, with the pluralities of RF coils secured to panels of fabric or other material forming the support structure 220. For example, the third coil array 206 may be secured to a first panel of fabric at the first side 250 of the support structure 220, and the fourth coil array 216 may be secured to a second panel of fabric at the second side 252 of the support structure 220. In the example shown by FIGS. 2A-2B, the first coil array 202 and second coil array 204 each include eight RF coils, and the third coil array 206 and fourth coil array 216 each include four RF coils. In other examples, the first coil array 202, second coil array 204, third coil array 206, and fourth coil array 216 may include a different number of coils relative to each other. For example, the third coil array 206 and fourth coil array 216 may each include eight RF coils, and the first coil array 202 and second coil array 204 may each include four coils. Other relative combinations of RF coils included by the first coil array 202, second coil array 204, third coil array 206, and fourth coil array 216 are possible.

Turning now to FIG. 4A, the first coil array 202 is shown removed from the support structure 220 in a first view 490 and the third coil array 206 is shown removed from the support structure 220 in a second view 492. Although the first coil array 202 and third coil array 206 are shown decoupled from the support structure 220 in the first view 490 and the second view 492 (respectively), the relative positions of the RF coils included by the first coil array 202 and third coil array 216 are the same as those shown by FIGS. 2A-2B. In other words, the RF coils of the first coil array 202 are coupled to the first cup 222 of the support structure 220 in the relative arrangement shown by first view 490, and the RF coils of the third coil array 206 are coupled at the first side 250 of the support structure 220 in the relative arrangement shown by second view 492. Although first coil array 202 is described herein with reference to the first view 490 of FIG. 4A, second coil array 204 includes a similar configuration of RF coils, materials, etc. Similarly, the third coil array 206 described herein with reference to the second view 492 includes a similar configuration of RF coils, materials, etc. as the fourth coil array 216. The third coil array 206 and fourth coil array 216 may be referred to herein as flank coil arrays, with the third coil array 206 positioned under a first arm of a patient and the fourth coil array 216 positioned under a second arm of the patient during conditions in which the RF coil assembly 200 is worn by the patient.

The RF coils included by the first coil array 202 may be mounted (e.g., coupled) to a flexible substrate (e.g., stretchable fabric, polymer, etc.). The flexible substrate may be fabricated from a material that is substantially transparent to RF and MR signals (e.g., a material that does not absorb, reflect, or otherwise interact with RF and MR signals). For example, the substrate may be fabricated from a cloth material or any other suitable material that is flexible to enable the RF coils to be repositioned relative to each other as described below with reference to FIGS. 5A-5B. In one example, the flexible substrate may be formed together (e.g., molded together) with the first cup 222 and/or support structure 220. In another example, the flexible substrate may be a same material as the material of the first cup 222 and/or support structure 220 and may be formed as a separate piece that is coupled to the first cup 222 (e.g., sewn, fastened, etc.). In other examples, the flexible substrate may be a different material than the material of the first cup 222 and/or support structure 220 and may be a separate piece that is coupled to the first cup 222 (e.g., sewn, fastened, etc.). The RF coils of the third coil array 206 may be coupled to a similar flexible substrate and/or may be formed together with the support structure 220. By coupling the RF coils to the flexible substrate and forming the flexible substrate as a separate piece relative to the support structure 220, the RF coils may be removed from the support structure 220 for cleaning, replacement, etc.

As described above, first coil array 202 includes a plurality of individual RF coils positioned in a symmetrical, circular arrangement, similar to an arrangement of petals of a lotus flower. The RF coils are local RF coils (e.g., similar to the local RF coils 14 described above with reference to FIG. 1) comprised of a generally flexible, flat, and electrically-conductive material (e.g., tin-plated copper). In the examples described herein, the first coil array 202 includes eight RF coils. Specifically, first coil array 202 includes first RF coil 400, second RF coil 402, third RF coil 404, fourth RF coil 406, fifth RF coil 408, sixth RF coil 410, seventh RF coil 412, and eighth RF coil 414. In other examples, the first coil array 202 may include a different number of coils (e.g., six, ten, twelve, etc.). Although the RF coils of the first coil array 202 are shown to be in an overlapping arrangement by the first view 490, in some examples the RF coils may not overlap each other. Additionally, in other examples, a size and/or shape of one or more of the coils may be different relative to other RF coils within the first coil array 202. Further, in some examples, the RF coils may be maintained at a fixed amount of overlap, and in other examples (such as the example described below with reference to FIGS. 5A-5B) the coils may be allowed to move relative to one another (e.g., to accommodate different breast sizes).

Various axes are shown by first view 490 in order to illustrate the relative positions of the RF coils described above. Specifically, a first axis 452 intersects a midpoint of the first RF coil 400 and a midpoint of the fifth RF coil 408, a second axis 454 intersects a midpoint of the second RF coil 402 and a midpoint of the sixth RF coil 410, a third axis 456 intersects a midpoint of the third RF coil 404 and a midpoint of the seventh RF coil 412, and a fourth axis 457 intersects a midpoint of the fourth RF coil 406 and a midpoint of the eighth RF coil 414. The RF coils are arranged symmetrically such that an amount of angle between midpoints of adjacent RF coils is a same amount of angle for each pair of adjacent RF coils. For example, a first angle 458 between the midpoint of the third RF coil 404 and the midpoint of the fourth RF coil 406 (e.g., an angle between the third axis 456 and the fourth axis 457) is a same amount of angle as a second angle 460 between the midpoint of the third RF coil 404 and the midpoint of the second RF coil 402. In one example, an angle (e.g., first angle 458, second angle 460, etc.) between adjacent coils within the first coil array 202 may be 45 degrees. In other examples including a different number of coils, the amount of angle between adjacent coils may be a different amount (e.g., 36 degrees with 10 RF coils, 30 degrees with 12 RF coils, etc.).

The third coil array 206 shown by the second view 492 of FIG. 4A similarly includes a plurality of RF coils positioned in a symmetrical arrangement. Specifically, the third coil array 206 includes a ninth RF coil 416, a tenth RF coil 418, an eleventh RF coil 420, and a twelfth RF coil 422. A fifth axis 462 and a sixth axis 464 are included by the second view 492 in order to illustrate the relative positions of the RF coils. The fifth axis 462 intersects a midpoint of the ninth RF coil 416 and a midpoint of the twelfth RF coil 422, and the sixth axis intersects a midpoint of the tenth RF coil 418 and a midpoint of the eleventh RF coil 420. Similar to the example described above, the RF coils of the third coil array 206 are arranged symmetrically such that an amount of angle between midpoints of adjacent RF coils within the third coil array 206 is a same amount of angle for each pair of adjacent RF coils. For example, a third angle 466 between the midpoint of the ninth RF coil 416 and the midpoint of the tenth RF coil 418 (e.g., an angle between the fifth axis 462 and the sixth axis 464) is a same amount of angle as a fourth angle 468 between the midpoint of the tenth RF coil 418 and the midpoint of the twelfth RF coil 422. In one example, an angle (e.g., third angle 466, fourth angle 468, etc.) between adjacent coils within the third coil array 206 may be 90 degrees. In other examples including a different number of coils, the amount of angle between adjacent coils may be a different amount (e.g., 120 degrees with 3 RF coils, 60 degrees with 6 RF coils, etc.).

The RF coils included by the third coil array 206 are local RF coils (e.g., similar to the coils of the local RF coils 14 shown by FIG. 1 and described above). In some examples, the RF coils of the third coil array 206 are similar to those included by the first coil array 202 (e.g., RF coils comprised of a generally flexible, flat, and electrically-conductive material, such as tin-plated copper). The RF coils may be coupled to flexible substrate, similar to the flexible substrate described above with reference to the first coil array 202. The flexible substrate may be formed from a substantially RF transparent material. For example, the substrate may be fabricated from a cloth material or any other suitable material that is flexible to enable the RF coils to be repositioned as described herein.

The flank coil arrays (e.g., third coil array 206 and fourth coil array 216) may enable imaging of a subject (e.g., subject 16 described above with reference to FIG. 1) in different directions (e.g., parallel imaging). Parallel imaging may result in reduced scan time, increased image resolution, artifact suppression, and attenuation of image noise. In a general sense, parallel imaging utilizes differences in sensitivities of individual coils within a coil array (e.g., third coil array 206) in order to reduce a number of gradient encoding steps required for imaging by supplementing coil spatial information with coil sensitivity information. Thus, in parallel MRI, an array of receiver coils with different sensitivities may be used to receive the MR signals in parallel, thereby facilitating combination of the received signals to reconstruct the full image via imaging approaches (e.g., protocols) such as SMASH (SiMultaneous Acquisition of Spatial Harmonics) and SENSE (SENSitivity Encoding). These parallel imaging approaches may be utilized with the RF coil assembly 200 in order to reduce a number of phase encoding steps and reduce imaging time.

FIGS. 3A-3B each show a second embodiment of an RF coil assembly 300. The RF coil assembly 300 shown by FIGS. 3A-3B differs from the RF coil assembly 200 shown by FIGS. 2A-2B in that the RF coil assembly 300 includes two coil arrays instead of four coil arrays. The two coil arrays are positioned to enclose the front and the sides of the patient in order to enable parallel scanning of the patient. In some examples, including two coil arrays configured to enclose the front and sides of the patient rather than four coil arrays may result in easier removal and/or coupling of the coil arrays to a support structure of the RF coil assembly 300 (as described below) for cleaning and/or replacement.

The RF coil assembly 300 includes a support structure 320 similar to the support structure 220 shown by FIGS. 2A-2B and described above. For example, the support structure 320 includes a first cup 322 having a circular frame 330 and an exterior surface 326, a second cup 324 having a circular frame 323 and an exterior surface 328, a front strap 314, a back strap 312, a first shoulder strap 308, and a second shoulder strap 310, similar to the first cup 222 having circular frame 230 and exterior surface 226, second cup 224 having circular frame 223 and exterior surface 228, front strap 214, back strap 212, first shoulder strap 208, and second shoulder strap 210 (respectively) shown by FIGS. 2A-2B and described above. Reference axes 399 are included by FIGS. 3A-3B for comparison of the views shown.

The RF coil assembly 300 includes a first coil array 302 and a second coil array 304. The first coil array 302 and second coil array 304 each include a plurality of RF coils. In one example, the RF coils are similar to the RF coils described above with reference to FIG. 5A. The first coil array 302 includes a plurality of RF coils coupled to the first cup 322 and the second coil array 304 includes a plurality of RF coils coupled to the second cup 324. The second coil array 304 additionally includes RF coils coupled to the support structure 320 at a first side 350 (e.g., first fabric panel) of the support structure 320 (e.g., similar to the first side 250 of support structure 220), and the first coil array 302 includes RF coils coupled to the support structure 320 at a second side 352 (e.g., second fabric panel) of the support structure 320 (e.g., similar to the second side 252 of support structure 220). In this configuration, the first coil array 302 may be utilized to receive MR signals from the patient at locations proximate to the first cup 322 and the second side 352, and the second coil array 304 may be utilized to receive MR signals from the patient at locations proximate to the second cup 324 and the first side 350. In this way, the first coil array 302 and second coil array 304 may enable MRI scanning of both the front and the sides of the patient (e.g., similar to the parallel imaging described above with reference to the RF coil assembly 200 shown by FIGS. 2A-2B).

The second coil array 304 is further illustrated by FIG. 4B and is shown removed from the support structure 320. Although the second coil array 304 is a single array configured to couple to the second cup 324 and the first side 350 of the support structure 320, the second coil array 304 includes sections that are individually similar to the coil arrays described above with reference to FIGS. 2A-2B. For example, the second coil array 304 includes a first section 470 similar to the first coil array 202 shown by the first view 490 of FIG. 4A and described above, and the second coil array 304 additionally includes a second section 472 similar to the third coil array 206 shown by the second view 492 of FIG. 4A and described above.

The first section 470 includes eight RF coils positioned in a symmetrical arrangement similar to the arrangement of the RF coils included by the first coil array 202 of the RF coil assembly 200, and the second section 472 includes four RF coils positioned in a symmetrical arrangement similar to the arrangement of the RF coils included by the third coil array 206 of the RF coil assembly 200. The first section 470 and second section 472 are coupled together via a third section 474. In the example shown by FIG. 4B, the third section 474 includes one RF coil. In other examples, the third section 474 may include a different number of RF coils coupling the first section 470 to the second section 472 (e.g., two RF coils, three RF coils, etc.). In yet other examples, the third section 474 may not include RF coils and may instead include a flexible substrate coupling the first section 470 to the second section 472 (e.g., similar to the flexible substrates described above with reference to the RF coils of the first coil assembly 200). In further examples, the third section 474 may include one or more RF coils and the flexible substrate coupling the first section 470 to the second section 472. By coupling the first section 470 to the second section 472 via the third section 474, the second coil array 304 may be more easily removed from the RF coil assembly 300 for cleaning, replacement, etc., and an amount of RF coils included by the second coil array 304 may be increased, thereby resulting in an increased quality of images produced by the MRI system.

FIGS. 5A-5B each show a cross-sectional view of the first coil array 202 of the RF coil assembly 200 shown by FIGS. 2A-2B and described above. FIG. 5B illustrates the first coil array 202 in an expanded state, and FIG. 5A illustrates the first coil array 202 in a non-expanded (e.g., contracted) state. In one example, the RF coils of the first coil array 202 may be biased toward the non-expanded state by the flexible substrate of the first coil array 202 and/or the material forming the first cup 222.

During conditions in which the RF coil assembly 200 is worn by a patient, the first cup 222 supports a breast of the patient. In order to conform a shape of the first cup 222 to a shape of the body of the patient, a distance between opposing coils of the first coil array 202 may expand from a first diameter 500 shown by FIG. 5A to a second diameter 502 shown by FIG. 5B. For example, the RF coils of the first coil array 202 may be coupled to the first cup 222 such that as the size of the first cup 222 increases in order to conform to the shape of the body of the patient (e.g., via the flexible material forming the first cup 222), a distance between opposing RF coils of the first coil array 202 increases (e.g., first diameter 500 between fourth RF coil 406 and eighth RF coil 414 increases to second diameter 502). In some examples, the first diameter 500 and/or second diameter 502 may be different amounts of length than the examples shown by FIGS. 5A-5B. The second diameter 502 is a non-limiting example and in some examples the first coil array 202 may be expanded from the first diameter 500 to a plurality of different diameters between the first diameter 500 and the second diameter 502. In this way, the RF coil assembly 200 may be utilized by patients having different body sizes, breast sizes, etc.

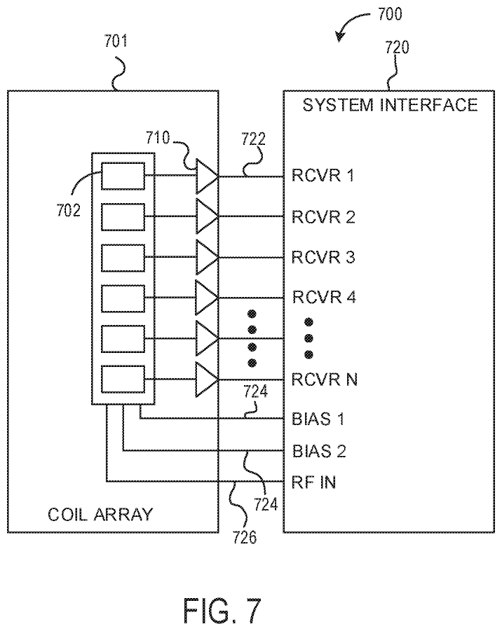

FIG. 7 shows a schematic diagram of an example of a signal-receiver section 700 of a magnetic resonance imaging (MRI) system (e.g., the MRI system 10 shown by FIG. 1 and described above) including an RF coil array 701. The signal-receiver section 700 may be referred to herein as a control circuit. The MRI system may be configured to reconstruct an image (e.g., an image of a patient) in response to signals received by a plurality of RF coils included within the RF coil array 701. In one example, RF coil array 701 may be similar to the coil arrays shown by FIGS. 2A-6 and described above (e.g., first coil array 202 shown by FIGS. 2A-2B, FIG. 4A, and FIGS. 5A-5B, first coil array 302 shown by FIGS. 3A-3B and FIG. 4B, etc.). Although the signal-receiver section 700 is described below with reference to the RF coil array 701, the signal-receiver section 700 may be utilized with any of the RF coil arrays described herein.

The RF coil array 701 includes a plurality of RF coils 702. In some examples, each of the RF coils 702 of the RF coil array 701 may be a loop and/or butterfly coil formed of a flexible, flat, and electrically-conductive material (e.g., tin-plated copper). The RF coils 702 are configured to detect MR signals. In some examples, two or more of the RF coils 702 may overlap with each other (e.g., may be positioned in a partially or fully overlapping arrangement relative to each other). Signals transmitted by the RF coils 702 to the MRI system are electrically isolated from each other via preamplifiers 710. The preamplifiers 710 additionally amplify the MR signals transmitted by the RF coils 702 (e.g., increase an amplitude of the MR signals). In some examples, the RF coil array 701 may configured to operate in a receive-only mode in which the RF coils 702 receive MR signals (e.g., from a body of a patient) and transmit the MR signals to the MRI system (e.g., to data acquisition unit 24 shown by FIG. 1 and described above), but the RF coils 702 do not transmit RF signals to the patient. In other examples, the RF coil array 701 may be a switchable array configured to operate in the receive-only mode or a receive-and-transmit mode in response to a selection made by an operator (e.g., user) of the MRI system (e.g., input at operating console unit 32 shown by FIG. 1 and described above). In the receive-and-transmit mode, the RF coils 702 may transmit RF signals to the patient and receive MR signals from the patient (as described above with reference to the MRI system 10 of FIG. 1). The RF coils 702 may transmit the received MR signals to the MRI system. Portions and/or an entirety of the signal-receiver section 700 may be referred to herein as a “system”.

The RF coil array 701 forms part of the multi-channel signal-receiver section 700 coupled to an MRI system. The signal-receiver section 700 includes a plurality of data channels (e.g., RCVR 1, RCVR 2, etc.). In some examples, the signal-receiver section 700 may include eight data channels. In other examples, the signal-receiver section 700 may include a larger or smaller number of data channels (e.g., six, ten, twelve, etc.), with the number of channels corresponding to the number of RF coils 702 utilized to form the RF coil array 701. In one example, the signal-receiver section 700 includes a multi-channel system interface 720 (e.g., a 1.5T System Interface). The multi-channel system interface 720 may be configured to include a relatively larger number of separate data channels 722 (e.g., RCVR 1, RCVR 2, etc.), with each of the data channels 722 coupled to separate RF coils (e.g., RF coils 702) of one or more RF coil arrays (e.g., RF coil array 701). For example, the multi-channel system interface may be configured to include 24 data channels, with each data channel coupled to separate RF coils of an RF coil assembly (e.g., RF coils included by the first coil array 202, second coil array 204, third coil array 206, and fourth coil array 216 of the RF coil assembly 200 shown by FIG. 1 and described above).

The system interface 720 may include a plurality of bias control lines 724 (e.g., BIAS 1 and BIAS 2) in order to control the switching of decoupling circuits (not shown), which may be controlled, for example, using a coil configuration file stored in computer memory of the MRI system and/or based on a user input. For example, based on a user input, a particular coil configuration file may be selected to control the RF coil array 701 in a particular imaging mode (e.g., a user-selected mode of operation via controls input on a control console, such as operating console unit 32 shown by FIG. 1 and described above). Additionally, an RF IN control line 726 may be included in electrical communication with, for example, a combiner (not shown) to control transmission of RF signals from RF coil array 701.

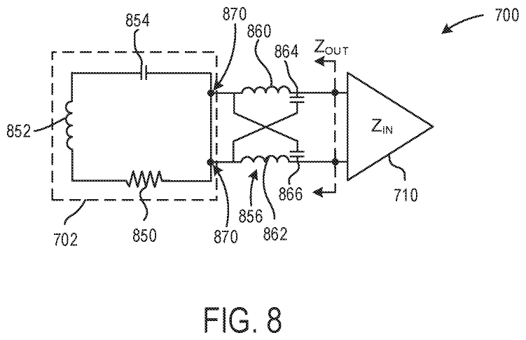

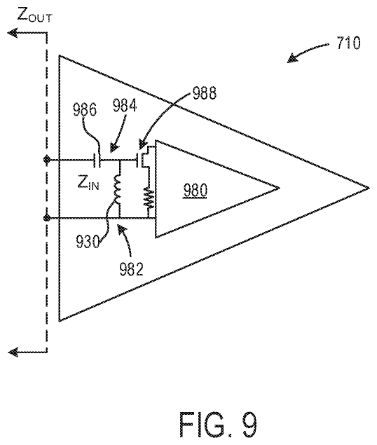

FIG. 8 shows a schematic diagram illustrating a portion of the signal-receiver section 700. The representative RF coil 702 shown by FIG. 8 is electrically coupled with corresponding pre-amplifier 710. Although a single RF coil 702 is shown by FIG. 8, each of the RF coils (e.g., RF coils of the RF coil array 701 shown by FIG. 7 and described above) may be in a similar configuration (e.g., may be similarly electrically coupled with corresponding pre-amplifiers, etc.). In some examples, the preamplifier 710 may have a relatively low input impedance. For example, a “relatively low” input impedance of the preamplifier 710 may be less than approximately 5 ohms at resonance frequency. The input impedance of the preamplifier 710 is defined by an inductor 930, which is shown in FIG. 9. Referring again to FIG. 8, the input impedance of the preamplifier 710 is represented by Z_{IN}. In other examples, the preamplifier 710 may have an input impedance of between approximately 1 ohm and approximately 3 ohms at resonance frequency. In further examples, the preamplifier 710 may have an input impedance of approximately 2 ohms at resonance frequency. It should be noted that for purposes of illustration, all of the capacitors are considered lossless and the inductors are represented with a series resistance. The input impedance of the preamplifier 710 may be referred to herein as a “preamplifier input impedance.”

The RF coil 702 include an RLC resonant circuit formed from a resistor 850, an inductor 852, and a capacitor 854. The RF coil 702 is connected in series to an impedance transformer 856. More specifically, the impedance transformer 856 is electrically connected between the RF coil 702 and the preamplifier 710. The impedance transformer 856 forms an impedance matching network between the RF coil 702 and the preamplifier 710. The impedance transformer 856 is configured to transform a coil impedance of the RF coil 702 into a source impedance of the preamplifier 710. The source impedance of the preamplifier 710 is represented in FIG. 8 by Z_{OUT}. The coil impedance of the RF coil 702 may have a value dependent on coil loading, coil size, field strength, and/or the like. Examples of the coil impedance of the RF coil 702 include, but are not limited to, amounts between approximately 2 ohms and approximately 10 ohms at 1.5T field strength, and/or the like.

In one example, the impedance transformer 856 includes a lattice-type balun. More specifically, the impedance transformer 856 includes two inductors 860 and 862 and two capacitors 864 and 866. The inductor 860 is connected in series to the capacitor 864, while the inductor 862 is connected in series with the capacitor 866. The inductor 860 and the capacitor 864 are connected in parallel to the inductor 862 and the capacitor 866. The arrangement of the lattice-type balun impedance transformer 856 produces a +/−90° phase shift. Each of the inductors 860 and 862 may be referred to herein as a “first” and/or a “second” inductor. The capacitors 864 and 866 may be referred to herein as a “first” and/or a “second” capacitor.

The impedance transformer 856 is configured to transform the coil impedance of the RF coil 702 into a relatively high source impedance Z_{OUT}. In some examples, a “relatively high” source impedance Z_{OUT} is at least approximately 100 ohms. Accordingly, the impedance transformer 856 is configured to transform the coil impedance of the RF coil 702 into a source impedance Z_{OUT} of at least approximately 100 ohms. In some examples, the impedance transformer 856 is configured to transform the coil impedance of the RF coil 702 into a source impedance Z_{OUT} of at least approximately 300 ohms, at least approximately 400 ohms, or at least approximately 500 ohms. Exemplary values for the inductors 860 and 862 include, but are not limited to, approximately 123.5 nH. Exemplary values for the capacitors 864 and 866 include, but are not limited to, approximately 51 pF.

The impedance transformer 856 also provides a blocking impedance to the RF coil 702. Transformation of the coil impedance of the RF coil 702 to a relative high source impedance Z_{OUT} may enable the impedance transformer 856 to provide a higher blocking impedance to the RF coil 702. Because the relatively high source impedance Z_{OUT} of the preamplifier 710 is greater than, for example, the conventional value of approximately 50 ohms, the reactance X of the inductors 860 and 862 and the capacitors 864 and 866 of the impedance transformer 856 are increased. For example, the reactance XC of each of the capacitors 864 and 866 and the reactance XL of each of the inductors 860 and 862 can be defined by the equation: XC=XL=√(R1×R2); where R1 is the coil impedance and R2 is the source impedance Z_{OUT}. Because the input impedance Z_{IN} of the preamplifier 710 is relatively low, the impedance transformer 856 forms a parallel resonance circuit that results in a higher impedance at an output 870 of the RF coil 702. As the reactances XC and XL increase, the blocking impedance increases because the blocking impedance is directly proportional to the values of XC and XL. The higher blocking impedance suppresses an increased amount of RF current along the RF coil 702, which may ultimately result in a higher SNR ratio because of fewer interactions between the RF coils of the RF coil array 701 and/or less correlated noise. Exemplary values for such higher blocking impedances include, for example, a blocking impedance of at least 500 ohms, and at least 1000 ohms.

The impedance transformer 856 is not limited to a lattice-type balun structure for transforming the coil impedance of the RF coil 702 into a relatively higher source impedance. Rather, any components and arrangement of the connections therebetween may be used to transform the coil impedance of the RF coil 702 into a relatively high source impedance, such as, but not limited to, other types of equivalent phase shift baluns, and/or the like.

FIG. 9 shows a schematic diagram illustrating the example preamplifier 710 shown by FIGS. 7-8. The preamplifier 710 is configured to accommodate the relatively high source impedance Z_{OUT} while providing the relatively low input impedance Z_{IN}. The input impedance Z_{IN} of the preamplifier 710 is defined by the inductor 930 of the preamplifier 710. The preamplifier 710 includes an amplifier 980 that receives MR signals from the corresponding RF coil 702 and amplifies the received MR signals. An input circuit 982 is electrically connected to the amplifier 980. The input circuit 982 is electrically connected to the output 870 (shown in FIG. 8) of the corresponding RF coil 702, via the impedance transformer 856 (shown in FIG. 8). The input circuit 982 is configured to transmit the MR signals from the corresponding RF coil 702 to the amplifier 980.

The input circuit 982 includes an impedance transformer 984, which includes a capacitor 986 and the inductor 930. The input circuit 982 also includes a field effect transistor (FET) 988 that is electrically connected between the impedance transformer 984 and the amplifier 980, for example as shown in FIG. 8. The impedance transformer 984 is electrically connected between the amplifier 980 and the corresponding RF coil 702.

The FET 988 has a relatively larger noise circle, which may be centered in the Smith Chart, for the FET 988 to yield a relatively lower noise figure. In other words, the FET 988 is capable of providing a relatively lower noise figure over a relatively broader range of source impedance Z_{OUT}. In some examples, a “relatively larger” size of the noise circle of the FET 988 is at least approximately 0.3 decibels. In other examples, the noise circle of the FET 988 has a size of at least approximately 0.6 decibels. The size of the noise circle of the FET 988 is dependent on the noise resistance RN of the FET 988. The FET 988 may have any value of noise resistance RN that provides a noise circle having a size of at least 0.3 decibels, such as, but not limited to, less than approximately 0.03 ohms, equal to or less than approximately 0.02 ohms, and/or the like. The location of the noise circle of the FET 988 within the Smith Chart is dependent on the optimum reflection coefficient of the FET 988. For example, the noise circle of the FET 988 may be located closer to the center of the Smith Chart (e.g., closer to being concentric) when the optimum reflection coefficient of the FET 988 is less than approximately 100 ohms. In some examples, the noise circle of the FET 988 is centered within the Smith Chart (e.g., concentric with the Smith Chart), and the FET 988 may have an optimum reflection coefficient of less than approximately 100 ohms. In other examples, the FET 988 has an optimum reflection coefficient of between approximately 40 ohms and approximately 60 ohms, for example approximately 50 ohms.

The electronics (e.g., preamplifiers 710, inductors 930, 852, 860, and 862, capacitors 854, 864, and 866, impedance transformer 856, etc.) described above with reference to the signal-receiver section 700 and FIGS. 7-9 are exemplary, and other electronics are possible. In one example, signal-receiver section 700 may include low source impedance preamplifiers instead of preamplifiers 710 and/or in addition to preamplifiers 710.

In order to make RF coils in an array sufficiently spatially distinct, and thus improve their signal-to-noise ratio (SNR) for accelerated imaging, it is common to leave gaps between neighboring (e.g., adjacent) coils within the array. This, however, has the drawback of increasing the coupling between the RF coils, which can in turn degrade performance. To overcome this, the RF coils in the array may be overlapped by an amount that minimizes the mutual inductance between neighboring coils. When such an array is employed for parallel imaging, the SNR decreases, because the geometry factor of the array has increased.

The RF coil assemblies described herein are bra-like coil assemblies that may increase patient comfort while increasing spatial resolution and reducing scan time during a breast MRI examination. Standard breast coil assemblies that include a middle divider may be particularly uncomfortable as the material of the coil is too hard and it is difficult for patients to keep a prone position and/or hold still for thirty minutes or more. Further, SNR performance of standard breast coils is relatively lower and may not meet clinical high spatial resolution requirements because of only eight receive channels and/or an increased distance between the breast and the coil surface. Further still, the scan time of breast MRI is relative longer than an MRI scan time of other parts of the body because of the multi-plan, bilateral breasts and single breast scans needed by a whole breast MRI examination. In standard breast MRI coil configurations, right to left direction acceleration is not an option because of a lack of available elements in this direction. Long scan times may not be suitable for some clinical applications. Additionally, standard coil assemblies may have reduced performance during chemical fat-sat (fat saturation) scans. B0 and B1 maps all show the obvious asymmetry. Standard breast coil assemblies additionally have reduced coverage of the axillary area. This may increase a difficultly of scanning axillary lymph nodes for breast cancer examination.

The technical benefits of the RF coil assemblies disclosed herein include providing comfortable, high performance breast coil assemblies that may be comprised of flexible and wireless “all” digital coils. Additionally, the coil assemblies disclosed herein may eliminate the skin-air interface, increase the regional B0 and B1 uniformity, and to increase clinical bad chemical fat saturation scan performance and right to left shading. The coil assemblies described herein may increase scan coverage of the axillary areas due to the additional elements in the right and left flanks of the patient. This may increase the detectable rate of the axillary lymph nodes for breast cancer MRI.

Increasing the number of RF coils per each breast (e.g., from 4 channels to 8 channels) enables the RF coils to be positioned more closely to each other and to anatomical structures that are to be imaged. The RF coil assemblies include adjustable straps in order to adapt to different body sizes. Additionally, the RF coils of each RF coil array may move relative to each other during conditions in which the material forming the cups is expanded or contracted, thereby enabling the RF coil assemblies to be utilized with an increased amount of body sizes. The RF coils coupled to the cups of the RF coil assemblies and the flanking RF coils positioned to surround the sides of the patient may enable parallel imaging of the patient in all directions, including right to left. As a result, a signal-to-noise ratio of signals transmitted by the coils may be increased. Additionally, parallel imaging via the RF coil assemblies increases an amount of the patient's body that may be scanned without repositioning the coils and/or the patient. In this way, scan time may be reduced and an imaging quality of axillary lymph nodes of the patient may be increased.

FIGS. 2A-2B, FIGS. 3A-3B, FIGS. 4A-4B, FIGS. 5A-5B, and FIG. 6 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space therebetween and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An RF coil assembly for a medical imaging device, comprising:
a first coil array housed in a first cup-shaped support structure;
a second coil array housed in a second cup-shaped support structure;
a third coil array flanking the first coil array; and
a fourth coil array flanking the second coil array.

2. The RF coil assembly of claim 1, wherein the first coil array includes a first plurality of RF coils positioned in a first symmetrical arrangement, where each RF coil of the first plurality of RF coils is angled by a first amount relative to each adjacent RF coil of the first plurality of RF coils.

3. The RF coil assembly of claim 2, wherein a distance between opposing RF coils of the first plurality of RF coils is adjustable from a first diameter to a second diameter.

4. The RF coil assembly of claim 2, wherein the third coil array includes a second plurality of RF coils positioned in a second symmetrical arrangement, where each RF coil of the second plurality of RF coils is angled by a second amount relative to each adjacent RF coil of the second plurality of RF coils.

5. The RF coil assembly of claim 4, further comprising a third plurality of RF coils positioned between the first plurality of RF coils and the second plurality of RF coils and coupling the first plurality of RF coils to the second plurality of RF coils.

6. The RF coil assembly of claim 5, wherein:
the second coil array includes a fourth plurality of RF coils positioned in the first symmetrical arrangement;
the fourth coil array includes a fifth plurality of RF coils positioned in the second symmetrical arrangement; and
a sixth plurality of RF coils positioned between the fourth plurality of RF coils and the fifth plurality of RF coils couples the fourth plurality of RF coils to the fifth plurality of RF coils.

7. The RF coil assembly of claim 1, further comprising one or more support straps supporting the first cup-shaped support structure, second cup-shaped support structure, third coil array, and fourth coil array, and wherein a length of at least one of the one or more support straps is adjustable.

8. The RF coil assembly of claim 1, wherein the first cup-shaped support structure is configured to receive a first breast of a patient and the second cup-shaped support structure is configured to receive a second breast of the patient.

9. The RF coil assembly of claim 8, wherein the third coil array is configured to be positioned under a first arm of the patient, adjacent the first breast, and the fourth coil array is configured to be positioned under a second arm of the patient, adjacent the second breast.

10. The RF coil assembly of claim 1, wherein the first coil array and the second coil array each comprise eight RF coils, and wherein the third coil array and fourth coil array each comprise four RF coils.

11. The RF coil assembly of claim 1, further comprising a preamplifier coupled to a first RF coil of the first coil array, the preamplifier comprising:
an amplifier; and
an impedance transformer configured to transform a coil impedance of the first RF coil to a source impedance of at least approximately 100 ohms.

12. A magnetic resonance imaging (MRI) system, comprising:
a gantry having a bore extending therethrough; and
a radio frequency (RF) coil assembly configured to be inserted into the bore, the RF coil assembly comprising a plurality of RF coils housed in a bra-like support structure, including a first set of RF coils housed in a first cup of the bra-like support structure, a second set of RF coils housed in a second cup of the bra-like support structure, a third set of RF coils housed on a first panel flanking the first cup, and a fourth set of RF coils housed on a second panel flanking the second cup.

13. The MRI system of claim 12, wherein the first set of RF coils includes eight RF coils, the second set of RF coils includes eight RF coils, the third set of RF coils includes four RF coils, and the fourth set of RF coils includes four RF coils.

14. The MRI system of claim 12, further comprising a control circuit configured to reconstruct an image based on signals received by the plurality of RF coils.

15. The MRI system of claim 14, wherein the control circuit is configured to reconstruct the image via a parallel imaging protocol.

16. A radio frequency (RF) coil assembly for a medical imaging device, comprising:
a bra-like support structure including a first cup, a second cup, a first panel flanking the first cup, a second panel flanking the second cup, and one or more adjustable straps;
a first RF coil array housed in the first cup and the first panel; and
a second RF coil array housed in the second cup and the second panel.

17. The RF coil assembly of claim 16, wherein the first RF coil array includes a first portion of RF coils positioned at the first cup and a second portion of RF coils positioned at the first panel.

18. The RF coil assembly of claim 17, wherein the first portion includes eight RF coils and the second portion includes four RF coils.

19. The RF coil assembly of claim 17, wherein the first RF coil array includes a third portion of RF coils, and wherein the third portion couples the first portion to the second portion.

20. The RF coil assembly of claim 19, wherein the third portion includes one RF coil.
